(19) United States
(12) Patent Application Publication
(10) Pub. No.: US 2009/0098015 A1
(43) Pub. Date: Apr. 16, 2009

(54) Ultrasonic Breathing and Respiratory System and Method

(76) Inventor: Mahesh C. Bhardwaj, State College, PA (US)

Correspondence Address: THE WEBB LAW FIRM, P.C., 700 KOPPERS BUILDING, 436 SEVENTH AVENUE, PITTSBURGH, PA, 15219, US

(21) Appl. No.: 12/251,653

(22) Filed: Oct. 15, 2008

Related U.S. Application Data
(60) Provisional application No. 60/979,980, filed on Oct. 15, 2007.

(51) Int. Cl. A61L 9/03

(52) U.S. Cl. 422/4

(57) ABSTRACT

A method and apparatus for treating the lungs by adding ultrasound energy to the inspiration air or gas.

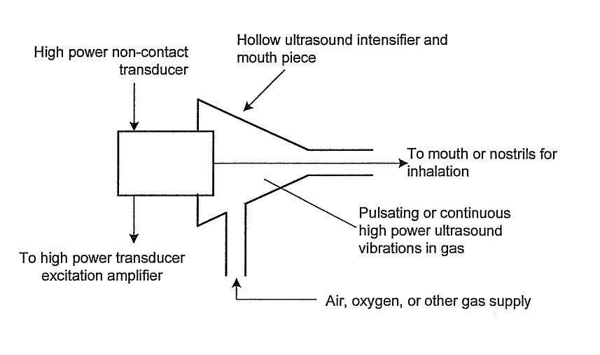

### BACKGROUND OF THE INVENTION

1. Field of the Invention

This invention relates to treatment of the lungs with inspiration air or gas energized with ultrasound.

2. Description of Related Art

Non-contact ultrasonic transducers, particularly the type described in my U.S. Pat. No. 6,311,573, are characterized by extremely high transduction efficiency in air or other gaseous media. When excited by a suitable electrical energy source, such as by a pulsed or continuous wave power amplifier, non-contact ultrasound transducers create immense acoustic pressure, also supplemented by heat generation. Intense acoustic pressure zones exist in axial and transverse regions of ultrasonic field in air/gas medium in which ultrasound is transmitted. In these, regions it is believed that intense heat is also generated due to air/gas molecular friction.

Evidence of these phenomena can be felt if one puts one's hand or any other part of the body one feels unusual sensation. Specifically, one gets a sensation that is not experienced in ordinary life events, such as those created by heat, pressure, or by chemical phenomena, for example, the feeling as if several needles are pounding the human skin and tissue, simultaneously generating unusual sensation of heat and pressure.

Similarly, if the surface of water or even solid material is exposed to such a field of ultrasound transmitted first through air, then one observes intense vibration of water or the material surface and its bulk. By raising or lowering the transducer in air with respect to its distance from the surface being irradiated by ultrasound, one observes maximum and minimum acoustic pressure zones (that can be observed with an unaided eye) which are separated by wavelength of ultrasound in air produced by the frequency of a given transducer.

By virtue of these observations, we decided to conduct an experiment to see if bacterial spores could be destroyed by using non-contact transducers. This experiment was successful and replicated several times and described in detail in a paper by Kelli Hoover, Mahesh Bhardwaj and Nancy Ostiguy entitled “Destruction of Bacterial Spores by High Power Non-Contact Ultrasound,” Mat. Res. Innovat., 6:291-295 (2002).

We have seen the observations described above by using non-contacting air or gas coupled transducers ranging in frequencies from 50 kHz to 500 kHz with active area diameters from 25 mm to 50 mm.

### SUMMARY OF THE INVENTION

Briefly, according to one embodiment of this invention, an apparatus for treating the lungs of a human or animal comprises an ultrasound intensifier and mouthpiece having an air/gas inlet, and a high powered non-contact ultrasound transducer, the ultrasound transducer directing ultrasound energy into the intensifier. The ultrasound transducer is preferably tuned to 50 to 500 kHz. The ultrasound intensifier preferably has a conical shell narrowing to a tubular conduit of cross-sectional size to enter a mouth or nostril and the ultrasound transducer is positioned at the wide end of the intensifier.

Briefly, according to another embodiment of this invention, a method of treating the lungs of a human or animal comprises increasing the energy of air or gas to be inspired with ultrasound and directing the air or gas to a month or nostril.

### BRIEF DESCRIPTION OF THE DRAWING

The drawing is a schematic of ultrasonic respirator or inhaler according to this invention.

### DESCRIPTION OF THE PREFERRED EMBODIMENTS

In the current invention, we envision application of high power ultrasound in air or other gases to clean lungs, to restore normal breathing, and other respiratory ailments. A schematic of such a system is shown in the drawing, which shows key components according to this invention and described as following:

Here, a non-contact air or gas-coupled transducer is excited by a suitable high power amplifier.

The transducer is connected to a conical hollow ultrasound intensifier that also acts as the mouthpiece (which can also be inserted in the nostrils), which is inserted in the mouth for normal breathing and inhalation.

This mouthpiece (which can also be inserted in the nostrils, that is, any part of the human or animal system that inhales air) can be connected to air, oxygen, or other gas supply for further enhancement of reaction, or it can be slightly open with a hole for normal ambient air flow.

By doing so, gas in the conical intensifier will be charged with pulsating or continuous high power ultrasound vibrations composed of acoustic pressure and molecular friction assisted heat.

By breathing ultrasonically charged gas through the mouth or through the nostrils, breathing passages and lungs will be cleaned, thus helping a person with breathing problems.

Having thus described my invention with the detail and particularity required by the Patent Laws, what is desired protected by Letters Patent is set forth in the following claims.

The invention claimed is:

1. An apparatus for treating the lungs of a human or animal comprising:
an ultrasound intensifier and mouthpiece having an air/gas inlet; and
a high powered non-contact ultrasound transducer, said ultrasound transducer directing ultrasound energy into the intensifier.

2. The apparatus according to claim 1, wherein the ultrasound transducer is tuned to 50 to 500 kHz.

3. The apparatus according to claim 1, wherein the ultrasound intensifier has a conical shell narrowing to a tubular conduit of cross-sectional size to enter a mouth or nostril and the ultrasound transducer is positioned at the wide end of the intensifier.

4. A method of treating the lungs of a human or animal comprising:
increasing the energy of air or gas to be inspired with ultrasound; and
directing the air or gas to a month or nostril.

5. The method according to claim 4, wherein the ultrasound has a frequency of 50 to 500 kHz.
